(19) United States
(12) Patent Application Publication
(10) Pub. No.: US 2013/0338016 A1
(43) Pub. Date: Dec. 19, 2013

(54) Method For Integrated Pathology Diagnosis And Digital Biomarker Pattern Analysis

(71) Applicant: Vala Sciences, Inc, (US)

(72) Inventors: Patrick M. McDonough, San Diego, CA (US); Jeffrey H. Price, San Diego, CA (US)

(73) Assignee: Vala Sciences, Inc., San Diego, CA (US)

(21) Appl. No.: 13/865,121

(22) Filed: Apr. 17, 2013

Related U.S. Application Data
(60) Provisional application No. 61/625,589, filed on Apr. 17, 2012.

(51) Int. Cl. G06F 19/18; G01N 33/58

(52) U.S. Cl. CPC G06F 19/18; G01N 33/582
USPC 506/8; 506/9; 435/7.1; 435/40.52

(57) ABSTRACT

A method of tissue analysis integrates a pathology diagnostic step (subjective human inspection of a stained tissue section or image of it) with one or more gene/biomarker tests to enable perform of both procedures side-by-side on the same instrument.

### PRIORITY

This application claims priority to US provisional application for patent 61/625,589, filed Apr. 17, 2012.

### STATEMENT OF GOVERNMENT INTEREST

An invention described herein was made in part with government support under Contract No. HHSN261201000088C awarded by the National Institutes of Health. The United States government has certain rights in this invention.

### BACKGROUND

The field is digital histocytometry. Digital histocytometry involves the use of an automated microscopy platform equipped with analytical tools to image cellular structures of an immunohistochemically-stained tissue sample mounted on a carrier such as a microscope slide and to measure features in the imaged cellular structures. The analytical tools are typically embodied in algorithms that perform image acquisition and measurement procedures. Immunohistochemistry denotes the use of immunohistochemical (IHC) dyes to label elements of biological tissue so as to visualize cellular structures in situ. IHC staining can utilize chromogenic or fluorescent labels. Fluorescence-based staining is frequently referred to as immunofluoresence (IF).

The increasing incidence of cancer in an aging population and the growing use of targeted cancer drugs requiring companion diagnostics using biomarkers are driving the rising need for automated testing and standardization. Cancer diagnostics are becoming increasingly complex due the increasing number of biomarkers upon which therapeutic decisions are based. Clinical pathologists must increasingly incorporate biomarker levels into their traditional cancer diagnoses and to do so must draw on test results that come from various modalities—tests on blood and tissue homogenates—in addition to visualization of tissue sections under the microscope. Classically, the pathologist diagnoses cancer by viewing tissue sections stained with hematoxylin and eosin (H & E), Papanicalou (Pap), and/or others (e.g., Table 1). New genes/biomarkers can be measured via immunohistochemistry (bright field or immunofluorescence labels) for cellular molecules including proteins, or chromogenic in situ hybridization (CISH) or fluorescence in situ hybridization (FISH) for nucleic acids (RNA or DNA). Many new tests involve processing a sample of the tissue in a manner that destroys the cellular structure and mixes intracellular components from many cells, which disassociates the test from the particular type of cell in the tissue section (cancer and various types of normal cells). The growing number of tests improves diagnosis and treatment, but also adds complexity to the pathologists' tasks, especially when such tests are performed using different techniques/instruments.

Therefore, one challenge is to create a test that enables both scoring of the key diagnostic molecules (genes, messenger RNA and proteins) in the tumor or surrounding normal tissue, directly in the same tissue section as the clinician uses to perform the classical initial diagnosis.

Another challenge is to create a test that enables both scoring of the key diagnostic molecules (genes, messenger RNA and proteins) in the tumor or surrounding normal tissue, directly in the tissue sections as those the clinician uses to perform the classical initial diagnosis.

### SUMMARY

A method integrates a pathology diagnostic step (subjective human inspection of the stained tissue section or image of it) with a gene/biomarker test(s) to enable the pathologist to perform both steps side-by-side on the same instrument.

A method labels these key molecules directly in the same cancer tissue sections as are labeled with classical subjective bright field microscopy stains such as H & E (see Table 1; http://en.wikipedia.org/wiki/Histology, which was sourced from Michael H. Ross, Wojciech Pawlina, (2006). Histology: A Text and Atlas. Hagerstown, Md.: Lippincott Williams & Wilkins. ISBN 0-7817-5056-3). The method includes a series of tissue preparation, labeling, automated microscopy (AKA, digital pathology), image analysis, and image and data display steps that result in an integrated display of the H & E image, the biomarker image(s) and the data for convenient and rapid analysis by the pathologist (and/or other physicians and clinical laboratory personnel).

This integrative tool makes pertinent information immediately available to the pathologist or cancer research scientist for rapid identification of both known and novel biomarker patterns. The tool enables rapid integration of sets of cancer biomarker pattern analyses into the tissue and cellular microscope-image viewpoint of the pathologist to create a multiplexed high throughput cancer biomarker histocytometry toolset, which will enable rapid visualization of complex patterns. Rapid visualization of cancer heterogeneity directly on images of the diagnostic hematoxylin and eosin (IA & E) tissue sections will speed biomarker discovery and translation of biomarkers to clinical use.

### BRIEF DESCRIPTION OF THE DRAWINGS

FIG. 1 is a magnified image of a breast tumor biopsy stained for H and HER2/neu, and converted from color to black and white (greyscale)

FIG. 2 is a set of magnified images of tissue sections derived from breast cancer cell lines labeled to show cell nuclei and immunofluorescently labeled biomarkersestrogen receptor (ER) and HER2/neu (HER2), cell image segmentation and a plot of the corresponding histocytometry data (cell-by-cell measurements).

FIG. 3 is a magnified image of tissue stained to show progesterone receptor expression in breast cancer tumor cells

FIG. 4 is a process flow diagram showing dual pathology diagnosis and biomarker pattern analysis.

FIG. 5 is a more detailed process flow diagram of FIG. 4.

FIG. 6 is a set of magnified images of prostate cancer tissue demonstrating side-by-side display and synchronized pan, scroll and zoom control of the immunofluorescence image and the H&E image of the same formalin-fixed paraffin-embedded tissue, along with simultaneous readout of the biomarker quantities.

FIG. 7A is a set of magnified images of prostate cancer tissue labeled and scanned using both H&E and immunofluorescence, which also further demonstrates the region selection (annotation of tissue sub-regions), along with corresponding cell segmentation and data plotting (see accompanying table) on the reactive stroma regions at various distances from the tumor

FIG. 7B shows further magnified views of the prostate tumor region of the area shown in FIG. 7A in which greyscale images of the fluorescence channel corresponding to biomarker AMACR and the fluorescence channel corresponding to cell nuclei (DAPI stain) are shown along with the cell numbering of the cells derived from image segmentation.

FIG. 8 includes a pair of plots that show performance of autofocus of an automated microscopy system with respect to measurements performed on a section cut from FFPE prostate cancer tissue and labeled with DAPI and for AMACR and ACTA2, along with greyscale images (converted from 3-channel color images) demonstrating the effects of some focus errors on image sharpness.

FIG. 9 is a set of magnified images of prostate cancer tissue showing In silica analysis of IHC labeling of Human Protein Atlas mages, which represent a process flow diagram for image segmentation of the biomarker and stroma regions.

FIG. 10 illustrates automated quantitation of “presence-of-tumor” from stroma biomarkers (“reactive stroma”) in Human Protein Atlas (HPA) images, along with biomarker expression dependence on the distance from the tumor.

FIG. 11 includes images of multiplexed labeling of the same tissue section for nuclei (with DNA dye BOBO-3), and immunofluorescently for ER, PR and HER2.

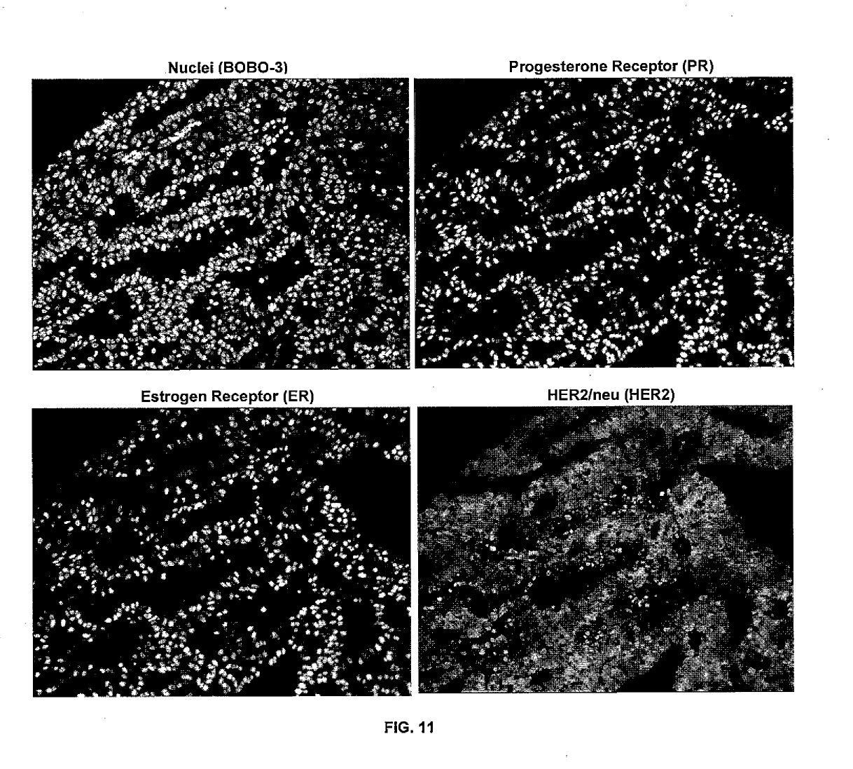

FIG. 12 illustrates quantification of the numbers of cells at different ER, PR and HER2 biomarker levels (intensities) in FFPET sections of breast cancer cell lines and cell counts of cells with various combinations of ER, PR and HER2 positivity/negativity (especially for detection of rare aggressive cell subpopulations in otherwise dominantly large populations of less aggressive cells).

FIG. 13 is a block diagram of a system for performing a computer-executed method of automated transient image cytometry according to the following specification.

### SPECIFICATION

Prostate Cancer Example. Prostate cancer will afflict one in six men in the U.S., resulting in almost 300,000 radical prostatectomies per year, causing substantial morbidity, anxiety and expense. There is growing evidence that many cases are treated more aggressively than necessary, and it is widely accepted that predictive methods of cancer progression would alleviate much of the untoward side effects and expenses (Andriole, G. L., et al. “Is there a better way to biopsy the prostate? Prospects for a novel transrectal systematic biopsy approach,” Urology 70, 22-26, 2007; Marks, L. S. & Bostwick, D. G. “Prostate Cancer Specificity of PCA3 Gene Testing: Examples from Clinical Practice,” Rev Urol 10, 175-181, 2008).

In contrast, aggressive prostate cancer is sometimes missed in patient biopsies because so little of the prostate is sampled. Up to 100,000 of these biopsied patients suspected to have prostate cancer have equivocal results that then lead to a repeat biopsy 3-12 months or more after the initial biopsy, creating a period during which tumor may progress. One group has developed a 114-gene (131 probe sets; 114 genes) Diagnostic Classifier that recognizes the presence-of-prostate cancer based on RNA expression changes in the stroma tissue adjacent to or near a site of tumor (i.e. the tumor microenvironment alone; Wang, Y., et al. “In silico Estimates of Tissue Components in Surgical Samples Based on Expression Profiling Data,” Cancer Res, 2010). Moreover, the expression changes in tumor-adjacent stroma are detected up to 11 millimeters from tumor thereby greatly extending the distance of detection beyond the needle biopsy which samples a cylinder of tissue with the diameter of 0.89 mm (Wang, Y., et al.). The gene expression changes of tumor-adjacent stroma are absent in normal prostate stroma. Thus, this represents an example of how measuring the patterns of biomarkers in the tissue sections could improve diagnosis and treatment.

Over- and under-treatment of prostate cancer both emphasize the difficulty physicians have diagnosing prostate cancer. Gene expression patterns have the potential to improve diagnoses, but even as new patterns are identified, the complexity of identifying patterns from multiple laboratory test sources may impede translation to the clinic. Integrated instrumentation and process tools enable the pathologist rapid viewing of gene patterns overlaid on diagnostic images of prostate cancer tissue sections—a novel high performance version of an automated digital pathology assistant. Automated microscopy is becoming increasingly commonplace for histology/pathology (Conway, C., et al. “Virtual microscopy as an enabler of automated/quantitative assessment of protein expression in TMAs,” Histochem Cell Biol 130, 447-463, 2008). These tools further enable cell-by-cell measurements (AKA “histocytometry”) as part of the automated digital pathology measurements. Desirably, these tools speed pattern analysis of this new information to simplify viewing of the complex and extensive data easily accessible to pathologists and cancer biologists.

Breast Cancer Example. Breast cancer is very common; approximately 180,000 new cases of invasive breast cancer were expected in 2008 in the US (the American Cancer Society web page www.cancer.org). Enormous health care costs are associated with breast cancer; for example, the economic burden to the state of California alone due to breast cancer was estimated at 1.43 billion dollars for 2001 (Max, W., Sung, H. Y. & Stark, B, “The economic burden of breast cancer in California,” Breast Cancer Res Treat, 2008). Breast cancer is heterogeneous, arising from different cell types, and has traditionally been classified as to the location of the tumors, tumor size, and the degree of invasiveness. Carcinomas, originating from epithelial cells, represent 90% of all human cancers, and are also the most common type of breast cancer. Breasts contain glandular lobules, which are the site of milk formation, and ducts through which milk flows. For in situ tumors, which have not yet invaded neighboring tissue, 80% are ductal carcinomas (DCIS=ductal carcinoma in situ) and 10% are lobular (LCIS). In situ tumors are considered Stage 0, and higher numerical values are assigned to tumors based upon size (e.g., Stage 1 is <1 inch), and invasiveness, and the potential for removal by surgery. Diagnosis of breast cancer involves mammogram screening, and about 2 to 3% of mammograms are followed by breast biopsy; the annual rate of breast biopsies is 62.6 per 10,000 women, annually, making breast biopsy a very common procedure. From the biopsies, prognostic information can be obtained, which provides the likely clinical outcome; predictive information is also obtained that guides the physician to choose the most effective therapies. Thus, biopsies are performed to diagnose the type and stage of the tumor and also to suggest potential therapeutic strategies. There is a very large effort within the biomedical community to advance and improve diagnostic procedures that can be applied to breast cancer biopsy material.

Breast cancer tumor specific gene expression: Using DNA microarray technology, “molecular portraits” of human breast tumors have been established in which >8000 genes were evaluated at the mRNA expression level, leading to 5 distinct tumor types; these include luminal type A, luminal type B (both luminal A and B are positive for estrogen receptors), basal-like, HER2/neu+, and normal breast. More traditional immunohistochemical staining and other techniques to detect protein expression have led to the identification of three proteins of particular interest. These are HER2/neu, estrogen receptor alpha (ERα), and the progesterone receptor (PR). (Typically here we refer to HER2/neu as “HER2” and ERα as “ER”, although “ER” can also refer to other subtypes) Use of the described tools is further illustrated using these biomarkers on breast cancer tissue, but it can be applied to tissue of any origin labeled with any set of biomarkers.

HER2/neu: An example breast cancer biomarker is HER2/neu, which is a member of the human epidermal growth factor receptor family; the ligand that may bind HER2/neu remains unidentified, however it is well known to be active as a heterodimerization partner for the other receptors. (Conway, C., et al. Virtual microscopy as an enabler of automated/quantitative assessment of protein expression in TMAs. Histochem Cell Biol 130, 447-463 (2008)). HER2/neu is overexpressed in 20 to 25% of breast cancers and patients with such tumors have a poor prognosis with shortened survival. Excess expression of HER2/neu upregulates mitotic pathways, leading to uncontrolled growth. Most commonly, HER2/neu protein overexpression is due to amplification of the HER2/neu gene. For example, increased expression of HER2/neu is associated with increased FISH (fluorescence in situ hybridization) signal, indicating gene amplification in 97% of samples. Located on chromosome 17q, most cells have two copies of the gene; however 50 to 100 copies of the gene may be found in certain tumors. It is becoming more and more routine among clinicians to assay the HER2/neu status of tumors by FISH. However, HER2/neu FISH is not, as yet, typically multiplexed with immunohistochemistry (IHC) to enable simultaneous assay of ER and PR in the same sample. FISH reagents are also much more expensive than IHC.

Establishing the HER2/neu expression status of a tumor helps predict the response of the tumors to various therapeutic strategies. Tumors that overexpress HER2/neu are likely more sensitive to anthracyclines; additionally, increased HER2/neu is associated with resistance to cytoxan-based treatments and increased resistance to tamoxifen for tumors that are also positive for the estrogen receptor. Perhaps most importantly, tumors that overexpress HER2/neu can be specifically treated with trastuzumab (Herceptin), a recombinant monoclonal antibody directed against HER2/neu. Trastuzumab features the HER2/neu binding region from a mouse monoclonal antibody fused to human IgG; it binds to HER2/neu and induces cytotoxicity of the tumor cell. Trastuzumab is indicated to be used in combination with paclitaxel as a first line treatment, or as a monotherapy for patients previously treated via chemotherapy. Thus, early detection of HER2/neu in breast tumors suggests the use of trastuzumab-based therapies, which significantly prolongs survival. However, the side-effects of trastuzumab include a low incidence of cardiotoxicity and heart failure and the cost of trastuzumab treatments are on the order of $100,000/year, so there are significant health and financial risks involved with overprescription of trastuzumab.

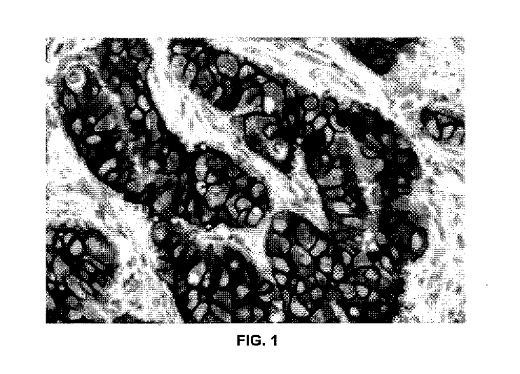

Referring now to FIG. 1 and Table 2, given the high incidence of HER2/neu overexpression in breast cancer, and the prognostic and predictive value of quantifying HER2/neu expression, the American Society of Clinical Oncology (ASCO) and the National Comprehensive Cancer Network strongly suggest that HER2/neu status be assayed in all newly diagnosed patients with invasive breast cancer (Perez, E. A. & Baweja, M, “HER2-positive breast cancer: current treatment strategies,” Cancer Invest 26, 545-552, 2008). Most commonly, HER2/neu is assayed by immunohistochemical analysis on formalin-fixed, paraffin-embedded tissue and the biopsies are scored 0, 1+, 2+ or 3+ on the basis of the staining intensity and cellular location. HER2/neu is present at the membrane and for strongly positive samples, is localized to the plasma membrane or borders between cells. Samples in which HER2/neu is absent (e.g., score=0) or very strongly expressed (score >2+) are typically diagnosed definitively by the pathologists; however, samples in the 1+ to 2+ range can be equivocal, due to the expression of HER2/neu in normal tissue, and due to variations in fixation, sample processing, differences in the antibodies used for the detection, and, likely differences in the scoring by individual pathologists. Variation in HER2/neu assay results is a prime concern in the area of breast cancer diagnosis. Indeed, in a retrospective study, biopsy material from patients enrolled in the NSABP B-31 (National Surgical Adjuvant Breast and Bowel Project), which were originally selected for participation in the study on the basis of a “3+” grading of HER2/neu staining, were retested and 18% failed to meet the 3+ criteria. By combining our analytical (quantitative) method with the images classically used by pathologists for diagnoses, we will provide the full range of biomarker quantity (e.g., positive for ER but at a low level) to better determine treatment. Digital analysis without a calibration standard has already been shown to improve diagnoses at the lower range of expression for ER. But this technique does not include the ability for the pathologist/diagnostician to view the classical clinically labeled (e.g., with H&E) image with the quantitative image and data.

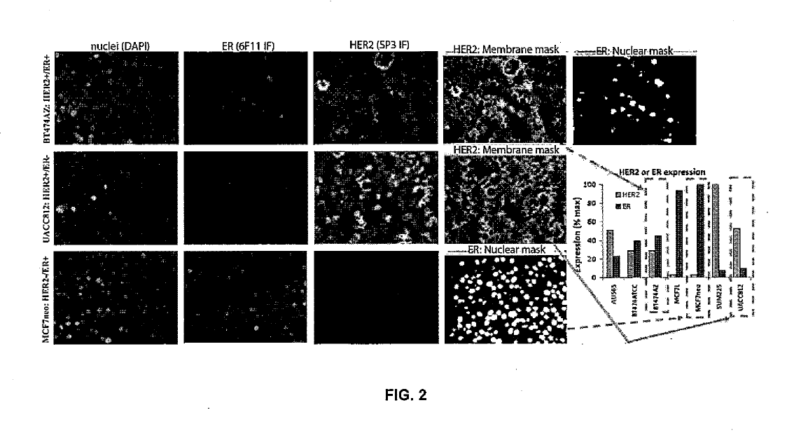

Estrogen receptors: Estrogen receptors are nuclear receptor family members primarily located within the nucleus (FIG. 2). That estrogen receptors (mainly the alpha subtype, ERα) participate in the etiology of breast cancer was first implied by the observation made in 1896 that removal of the ovaries would lead to a regression in advanced breast tumors. Estrogen stimulates mitosis in breast epithelial cells, and elevated blood estrogen concentration increases the risk of breast cancer; furthermore, estrogen hormone therapy, proscribed to post-menopausal women, increases the risk of breast cancer. Anti-estrogens are one of the most important therapeutic classes for breast cancer. Tamoxifen, the “flagship” anti-estrogen, was approved as an adjuvant therapy in 1985 and as a chemopreventive agent in 1999. For patients with ERαpositive breast tumors, tamoxifen reduces the risk of death by 28% and the incidence of contra lateral breast cancer by 47% (Jordan, V. C., Gapstur, S. & Morrow, M, “Selective estrogen receptor modulation and reduction in risk of breast cancer, osteoporosis, and coronary heart disease,” J Natl Cancer Inst 93, 1449-1457, 2001). Tamoxifen is not ideal, however, as it increases the incidence of endometrial cancer, stroke, pulmonary emboli deep vein thrombosis, and cataracts (Lee, W. L., Cheng, M. H., Chao, H. T. & Wang, P. H, “The role of selective estrogen receptor modulators on breast cancer: from tamoxifen to raloxifene,” Taiwan J Obstet Gynecol 47, 24-31, 2008); tamoxifen acts as an estrogen antagonist in most reproductive tissues, but can act as a partial ERα agonist in certain contexts, which may explain the variety of side effects. The development of “Selective estrogen receptor modulators” (SERMs) and “pure estrogen antagonists” which lack the partial agonistic action of tamoxifen, are emerging classes of therapeutic agents against breast cancer. Another emerging class are the aromatase inhibitors, which inhibit peripheral estrogen synthesis.

Approximately ⅔ of human breast tumors exhibit elevated ERα expression. Most low-grade tumors are ERα positive, and the presence of elevated ERα is highly correlated with sensitivity to tamoxifen. As many as 50% of patients with ER-positive metastatic tumors initially achieve positive therapeutic effects with tamoxifen; unfortunately, virtually all of these patients eventually relapse while taking tamoxifen and eventually die from the disease, as the tumor cells become resistant to tamoxifen. In cells derived from breast tumors, continuous exposure to tamoxifen in culture or xenografts (in which human breast cancer cell lines are implanted into mice) leads to increased expression of HER2/neu. In studies in which tumors were assayed before and after long-term tamoxifen exposure, ERα was lost from the tumors approximately 17% of the time; in the other cases, the tumors were unresponsive to tamoxifen even though ERα was still present, suggesting a loss of coupling between ERα and downstream effectors. Tumors that are negative for ERα are more aggressive than tumors that express ERα, and there are fewer therapeutic options for their treatment.

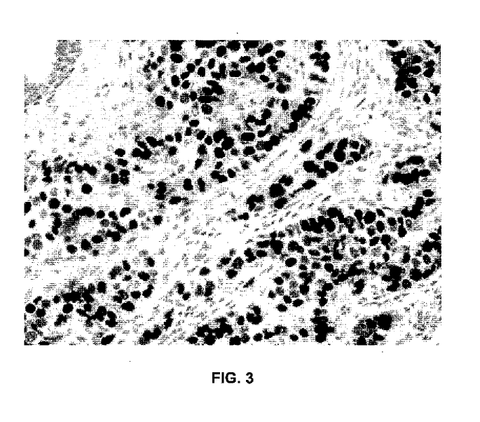

Progesterone receptors: Progesterone receptors (PRs) are also members of the nuclear receptor subfamily (FIG. 3). There are two isoforms (PRA and PRB), and both isoforms contribute to normal breast cell biology and the etiology of breast cancer; both isoforms are commonly detected, together, via immunohistochemistry, using antibodies that will bind to both PRA and PRB. The College of American Pathologists and ASCO recommend that PR should be assayed in all primary breast cancers. The expression of PR is dependent on ERα signaling. Thus, the expression of PR is evidence that the ERα signaling pathways are functioning, and, in general, tumors with PR are sensitive to tamoxifen. Tumors which are positive for PR, but negative for ERα are rare, and might represent “false negative” results in the ERα assay and such tumors may be responsive to tamoxifen. In contrast, tumors that are positive for ERα, yet negative for PR, may be tumors in which downstream signaling from ER has been interrupted, and tamoxifen may be ineffective. Recent results, utilizing profiling of RNA expression support the hypothesis that ER+/PR− tumors are very distinct from ER+/PR+ tumors in terms of gene expression, and they are known to represent a highly aggressive tumor type with poor prognosis.

Overall, the relationship between HER2/neu, ERα, and PR expression is very complex and not fully understood. There is considerable “crosstalk” between the ERα, PR, and HER2/neu pathways. For example, a subset of ERs likely associates with the plasma membrane and interacts with and activates growth factor receptors, including HER2/neu. Additionally, ERα is phosphorylated by growth factor-dependent pathways. HER2/neu has been postulated to participate in the development of resistance to tamoxifen. In certain xenograft models, exposure to tamoxifen increases HER2/neu expression and HER2/neu expression increases further, when the tumors become resistant to tamoxifen. HER2/neu also potentially increases the agonistic activity of tamoxifen.

Relatedly, “triple negative” breast cancer tumors are tumors that are scored as negative for HER2/neu, ERα, and for PR. Basal carcinomas, which often express genes found in normal basal/myoepithelial cells of the breast, are also often negative for HER2/neu, ERα, and PR. Both triple negative tumors and basal carcinomas are aggressive tumors associated with poor prognosis (shortened patient survival). For the triple negative tumors most patients die within 5 years.

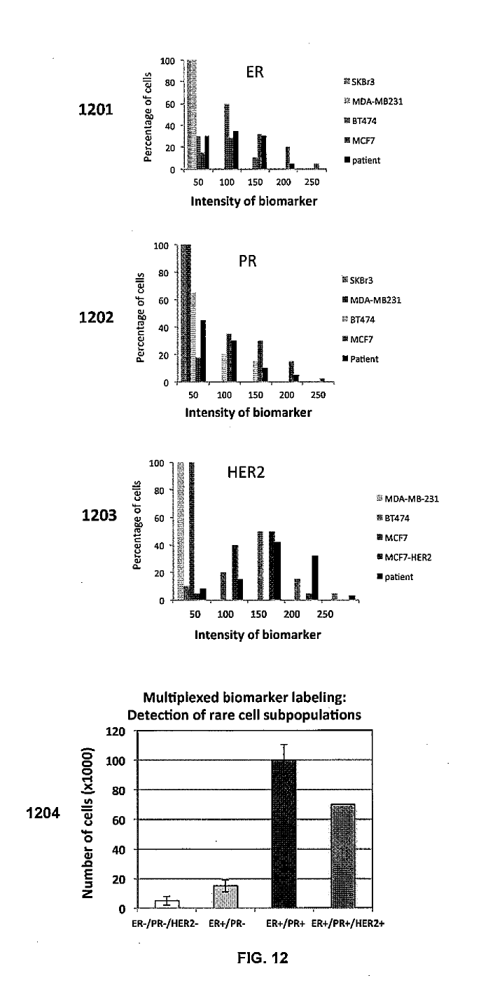

It is thus a further goal to utilize multiplexed labeling of biomarkers in the same tissue section cell-by-cell such that individual cells with aggressive combinations of biomarkers such as ER+/PR− and HER2−/ER−/PR− (FIG. 12) can be detected. Current single-label per tissue section techniques typically utilized for clinical diagnostics have thus far prevented the clinical trials that could characterize the utility of detecting rare aggressive subpopulations of cells in tissues expressing predominantly less aggressive biomarker patterns.

The significance of these breast cancer biomarkers, coupled with the challenges of the current subjective scoring of their expression and the opportunity for detection of rare aggressive subpopulations, motivate development of a convenient automated method of measuring biomarker levels and viewing the quantitative information alongside the clinical labels (e.g., H&E). An integrated histocytometry system automatically analyzes the expression of HER2/neu, ER, and PR, and simultaneously displays images scanned using the classical clinical label on the same tissue section. Methods for sequentially immunofluorescently labeling the same tissue section using antibody “stripping” techniques, and sequentially using different tissue sections for each combination of biomarkers, each with its own calibration standard (U.S. 61/620,897), precedes labeling with bright field stains such as H&E.

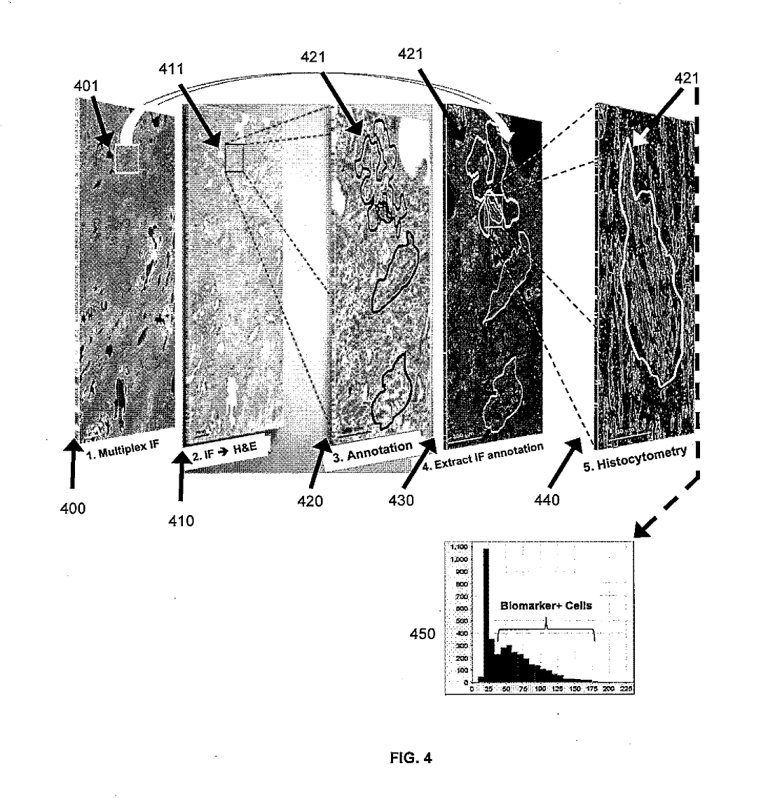

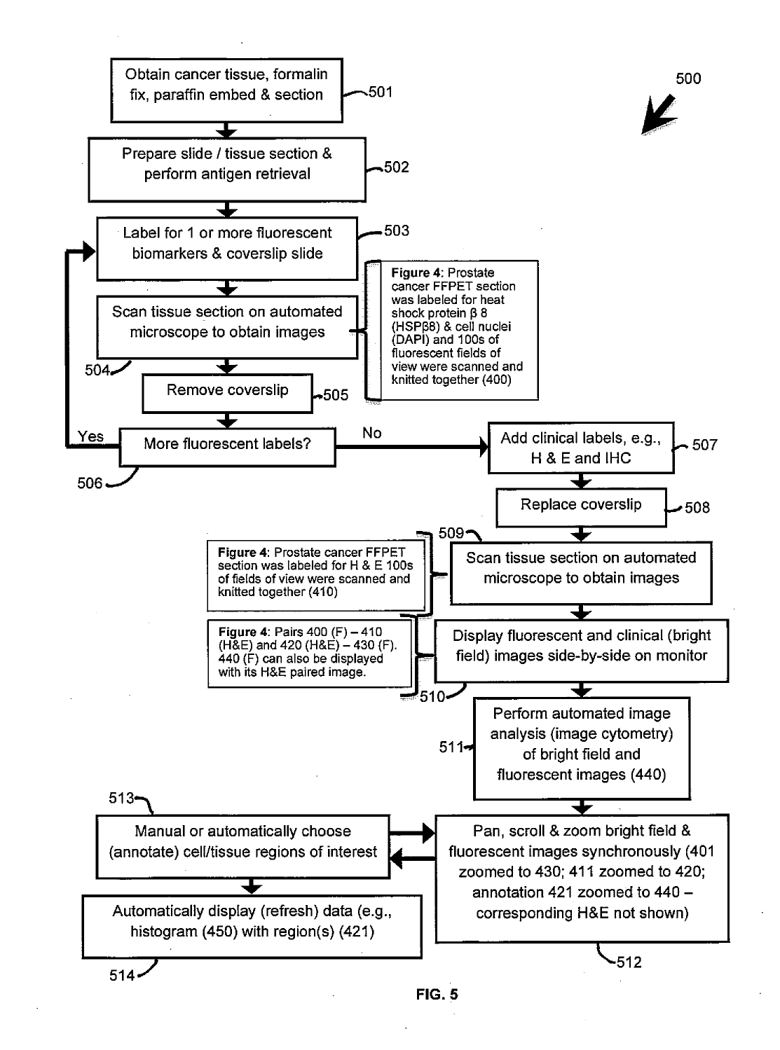

Alternatively, H is typically non-fluorescent (e.g., VECTOR Hematoxylin QS, http://www.vectorlabs.com/catalog.aspx?prodID=18), and can be used to label the tissue prior to fluorescent labeling, enabling both bright field and fluorescent labels to be combined at the same time, and thereby simplifying the steps of FIGS. 4 and 5 by removing the need to remove the coverslip after fluorescent scanning prior to the bright field scan.

Immunohistochemical (INC) detection of cancer biomarkers: Samples of tumor material are collected (biopsies, or the tumors, themselves); the samples are then fixed, dehydrated and cleared (through use of graded ethanol solutions and xylene), embedded in paraffin, and cut into sections (typically 5 μm thick) which are mounted on a glass slides for further processing. For labeling, the slides are “deparaffinized”, rehydrated, and incubated with a primary antibody that specifically binds to the protein of interest (e.g., an antibody against HER2/neu) in samples that are typically sections of tissue (the derivation of “histo”). The primary antibody is then recognized by a secondary antibody that is conjugated to an enzyme (e.g., horse radish peroxidase (HRP)) that catalyzes conversion of a substrate (“chemical”, e.g., 3,3′-diaminobenzidine (DAB)), to produce a colored precipitate (brown for DAB) or stain in the sample. The sections are also typically stained with additional reagents (e.g., hematoxylin, or hematoxylin+eosin) to generally visualize cellular structures. The slides are then viewed and scored by a pathologist using traditional light microscopy. The disadvantages of clinical IHC are that it is not stoichiometric and it has inherently low dynamic range compared with fluorescence. For these reasons, virtually all IHC based methods (e.g., gels etc.) have moved to fluorescence to create analytical measurements; clinical histology is ripe for this same transition.

Difficulties in tumor classification: The IHC techniques are well established, but are done with subtle differences in protocol, lab to lab, and variations in quantification of HER2/neu, ER, and PR in samples originating from breast tumors is of considerable ongoing concern. For HER2/neu, up to 20% of the current testing may be inaccurate, and there may be a higher degree of discrepancies in scoring at small volume “local” laboratories, vs. larger-volume “central” laboratories. Breast cancer tumor and biopsy material (as well as slides for pathological evaluation, in general), are processed by automated staining systems. Furthermore, with the explosive growth in digital microscopy technology (microscopes interfaced to digital cameras) “slide scanners”, which image IHC slides using light microscopy techniques are currently marketed from several companies (e.g., Dako, whose ACLS instrument was purchased from Clarient in 2005, and Aperio purchased by Leica Biosystems). Image analysis algorithms have also been developed for quantifying protein expression in images of IHC obtained by slide scanners. Aperio, for example, has obtained FDA clearance for a process specifically designed for analysis of images obtained from HER2/neu IHC-processed slides, which “is intended to be used as an aid to pathologists in detecting and quantifying HER2 protein expression from digital slide images created by Aperio's slide scanning systems” (from the Aperio web site). IHC methodology is most often used to specifically visualize a single protein in the sample against a background in which the sample is stained in a non-specific manner to visualize cell structures. While it is possible in some cases to add a second protein, (e.g., utilizing a second pair of primary and secondary antibodies, utilizing a different colorimetric reagent for the second protein species), the images are difficult to analyze in a digital quantitative fashion, due to spectral overlap between the stains and labels. Multiplexing of multiple biomarkers is thus easier with fluorescent labels.

“False negatives” for ER and HER2/neu: Some degree of disagreement between results reported for different pathologists or laboratories for the same material is inevitable as the sample evaluation depends upon visual interpretation which can be influenced by the skill or experience level of the pathologist or other human factors. Interesting in an extensive study featuring “blinded” review of the pathological evaluation of surgical samples representing a variety of tissue types, the overall rate of disagreement was 6.9%, and the disagreement rate was highest (13%) for breast cancer. The central problem to address is that the likely therapeutic strategy for a patient is now being determined by the HER2/neu and ER (and PR) status of the tumors; thus, “false negatives” represent a pool of patients that will not receive the appropriate therapies that are available, and will have an increased mortality as a result.

For HER2/neu, separate slides obtained from the same specimen can be evaluated HER2/neu status by two techniques (IHC and by FISH). However, for both techniques, equivocal results are commonly found (e.g., IHC grades for HER2/neu less than or equal to 2, or HER2/neu:CEP17 FISH ratios of 1.8 to 2.0). Part of the difficulty for HER2/neu is that it is endogenous, so there is a certain level of expression by the normal cells. In general, though, there is very high correlation between FISH and IHC, and the availability of the two techniques provides a “fail-safe” to the diagnosis, which leads to decisions regarding the use of Herceptin as a therapeutic strategy.

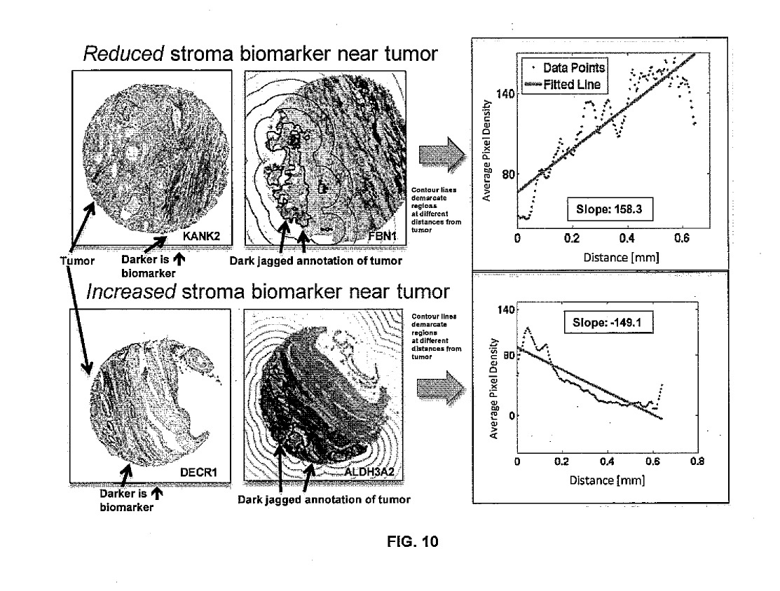

Optimal quantification of breast tumor ER, PR and HER2 status is a topic of intense research interest for the breast cancer community; e.g., the ER status of the tumor influences the decision by the physician to prescribe tamoxifen and related medications. Importantly, there are ongoing efforts to standardize IHC techniques for biomarker determinations, and the identification of positive cells can be highly dependent upon the antibody used for IHC. Furthermore, there are ongoing efforts to detect ER status via PCR techniques. There is a clear need for quantitative microscopy-based assays for ER, PR and HER2 since image-based techniques conserve the spatial relationship of cells within the tumor, which is important for diagnosis (FIG. 10). Fluorescence microscopy, with its greater degree of dynamic range for detection and quantification, and digital imaging, provide the best solution to the ER and PR, and general cancer biomarker quantification problem.

Immunofluorescence detection of cancer markers: Immunofluorescence (IF) is similar to IHC in the initial sample processing steps (e.g., fixation and antigen retrieval), however, in IF, the secondary antibodies are conjugated to fluorescent compounds. Also, fluorescent stains are used to stain the tissue for certain cellular features (e.g., DAPI for nuclei). The fluorophores have distinct excitation and emission spectra, and they are bright enough so as not to require enzymatic amplification, which results stoichiometry of brightness as a function of expression. The samples are imaged on fluorescent microscopes that are outfitted with optical band pass and dichroic filters that specifically define specific fluorescent channels (e.g., 360 nm/465 nm for excitation and emission of DAPI). With appropriate fluorophores and filter sets, images obtained for a specific channel will visualize only a single structure or protein of interest. For example, a sample can be labeled for DAPI in the blue (UV) fluorescence channel, HER2/neu in the green fluorescence channel (ex=492 nm, em=535 nm) and ER in the near red channel (ex=572, em=630). In typical fluorescence digital microscopy workstations, up to 4 channels are routinely employed (blue, green, red, far-red), meaning that 4 structures or proteins can be independently imaged and analyzed from the same sample. Since digital cameras are used to acquire the images, the intensity of each pixel is represented by a digital value (e.g., 0 to 255 for 8-bit images, 0 to 64000 for 16-bit images). For each field of view, a sample is typically imaged, sequentially, in each of the fluorescence channels.

High Content Analysis: High Content Analysis (HCA) refers to the fact that cell-based images contain a potential wealth of information, relating to cell size, nuclear shape and DNA staining intensity, etc. HCA most often utilizes digital fluorescence microscopy because of the aforementioned advantages in the quantification of pixel intensities associated with specifically-labeled cellular structures or proteins to produce high contrast black-and-white images that lack the background of IHC images. Algorithms have been developed, for example, to identify the nuclei and plasma membrane, and nuclei can be identified from either a fluorescent DNA label or H, which can be combined with fluorescent labeling. IF imaging techniques have previously been applied to HER2/neu and ERα in breast cancer tumor material (Dolled-Filhart, M., et al., “Quantitative in situ analysis of beta-catenin expression in breast cancer shows decreased expression is associated with poor outcome,” Cancer Res 66, 5487-5494, 2006; Giltnane, J. M., et al., “Comparison of quantitative immunofluorescence with conventional methods for HER2/neu testing with respect to response to trastuzumab therapy in metastatic breast cancer,” Arch Pathol Lab Med 132, 1635-1647, 2008), though only a single receptor (e.g., HER2/neu or ERα) was characterized, per assay, the cell-by-cell “histocytometry” analysis (AKA image cytometry) was not performed, and there was no on-slide analytical standard. With dual HER2-ER IHC staining of tissue sections from 148 DCIS patients, 14 exhibited coexpression in the same cells, all of which were of high nuclear grade (Dako Envision Double IHC kit; Collins, L. C. & Schnitt, S. J., “HER2 protein overexpression in estrogen receptor-positive ductal carcinoma in situ of the breast: frequency and implications for tamoxifen therapy,” Mod Pathol 18, 615-620, 2005), but these were analyzed manually. Four channel labeling of formalin-fixed, paraffin-embedded, breast cancer tumor tissue sections has recently been achieved, in which the visualized structures and proteins were nuclei (with DAPI), CK8/18, ERα, and vimentin, and in this study the samples were stored and retested 270 days after sample processing, with little or no loss of image quality (Robertson, D., Savage, K., Reis-Filho, J. S. & lsacke, C. M., “Multiple immunofluorescence labeling of formalin-fixed paraffin-embedded (FFPE) tissue,” BMC Cell Biol 9, 13, 2008).

Our tools differ from other semi-automated IHC readers (e.g., Aperio ScanScope with HER2 FDA approval October 2007, Aperio Receives FDA Clearance for HER2 Image Analysis Application, http://www.aperio.com/newsevents/oct_{—}17_{—}07.asp; and Dako ACIS FDA approved FDA cleared instrument and algorithms for HER2, ER and PR on the Dako ACIS II Automated Cellular Imaging System http://pri.dako.com/28247_{—}080505_acisii_brochure.pdf) in that the diagnostic and analytical labels are imaged on the same tissue section, enabling the diagnostician to view the relationship between, e.g., higher grade (likely more aggressively metastatic) cells in the classical bright field images (H or H&E) and their expression of the biomarkers using fluorescence in the same cells. Our approach is also different in that a process executed at least in part by an automated cytometry system will fully automatically perform cell-by-cell image cytometry and the test will be fully automated against a calibration standard to produced a score that the pathologist will review to produce a final score (U.S. 61/620,897), enabling the diagnostic-analytical combination to be carried out on each cell. In contrast, the ScanScope and ACIS processes require manual calibration of the colors to select only regions of the image containing many cells for subsequent digital scoring, and do not contain on on-slide analytical standard (reference our on-slide calibration patent application). Cell-by-cell cytometry also detects and measures multiple biomarkers (via multiplexing) in the same cell, which enables identification of particular patterns of biomarkers associated with more or less aggressive cells, thereby enabling detection of aggressive rare cell subpopulations (FIG. 12).

Interesting examples of analysis of breast cancer cells and tumor sections have been performed previously using quantitative fluorescence microscopy techniques, in particular by Rimm and colleagues using the AQUA system. Of particular interest are studies by this group in which it has been demonstrated that the predictive information regarding patient survival and HER2/neu labeling can depend greatly upon the concentration of antibody used to detect HER2/neu (though detection of ER was less prone to this effect). This underscores the inherent variability that precludes standardization across different labs with current techniques, which cannot correct for differences in labeling techniques. Also of high interest, was the quantification by these workers of the heterogeneity of ER expression in adjacent sections of breast cancer tumors. These results emphasize the need for continuing development of quantitative assays for HER2/neu, ER, and PR, especially multiplexed assays with an analytical calibration standard in which these biomarkers can be assayed quantitatively on the same section, and, indeed, on the same exact cells (on a cell by cell basis), as is the goal of assay development in the present project, to produce detailed results standardized between different patients and across different labs. According to methods described herein, these quantitative advantages are combined directly with the classical diagnostic images on the same tissue section to improve diagnoses.

Formalin Fixed Paraffin Embedded (FFPE) Prostate Cancer Tissue Section: Immumofluorescence and H&E on the Same Tissue Section

Integrated Diagnostic Images and Histocytometry Biomarker Patterns. Reading a report of RNA expression from rtPCR of tissue samples makes it difficult to discern the relationship between the cancerous appearing cells as a pathologist views them in an H&E section and the biomarker data. Furthermore, data from mixtures of cells cannot be used to identify biomarker patterns at cell-by-cell resolution in the tissue and cannot be used to identify “pure pattern biomarkers” like translocation/redistribution (such as by □-catenin from the membrane to the nucleus).

The tools and methods described herein enable display of H&E, immunolabeling and biomarker pattern summaries together to speed identification of new biomarker patterns and clinical translation (FIGS. 4-7).

In FIG. 4, a method of integrating diagnostic images with biomarker patterns for presentation is represented by low magnification images (comprising typically hundreds of magnified fields of view) of immunofluorescent (IF) labels (400) and H&E (410). A user selects a region (401) in the IF image & 411 in the H&E image) and zooms both synchronously (430 and 420, respectively), annotates (manually segment) a region or regions (421), further zooms on the annotated region (440), if needed, and with annotation is automatically presented with a display of the histocytometry (cell-by-cell data, 450).

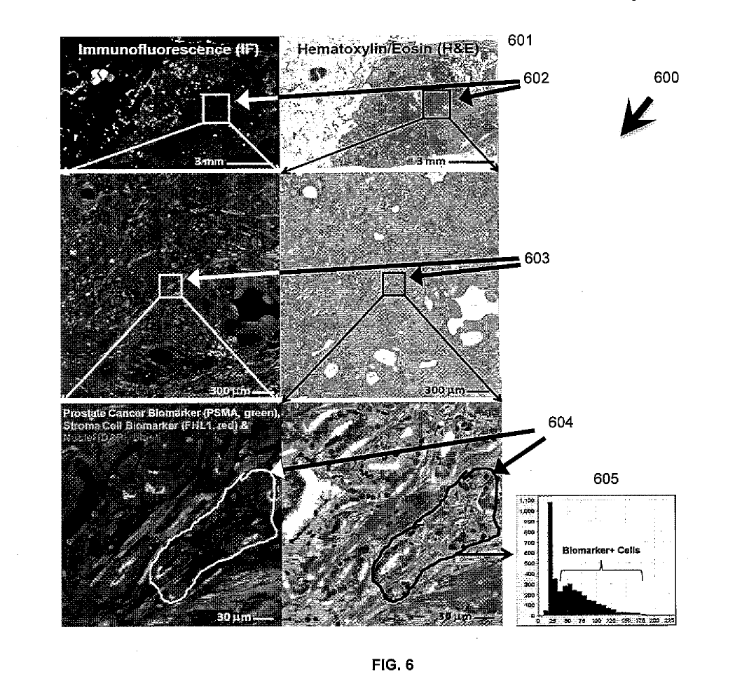

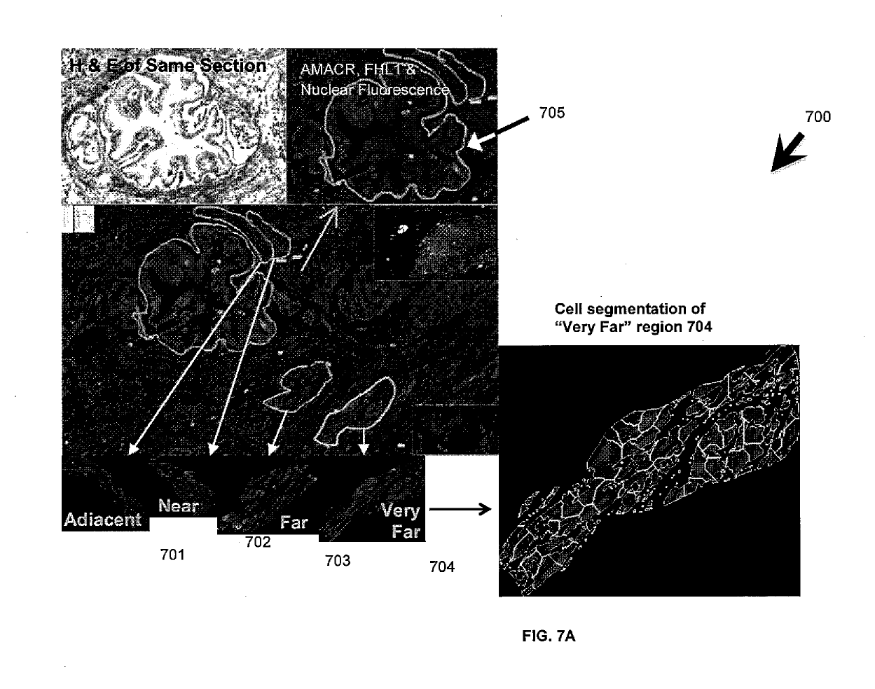

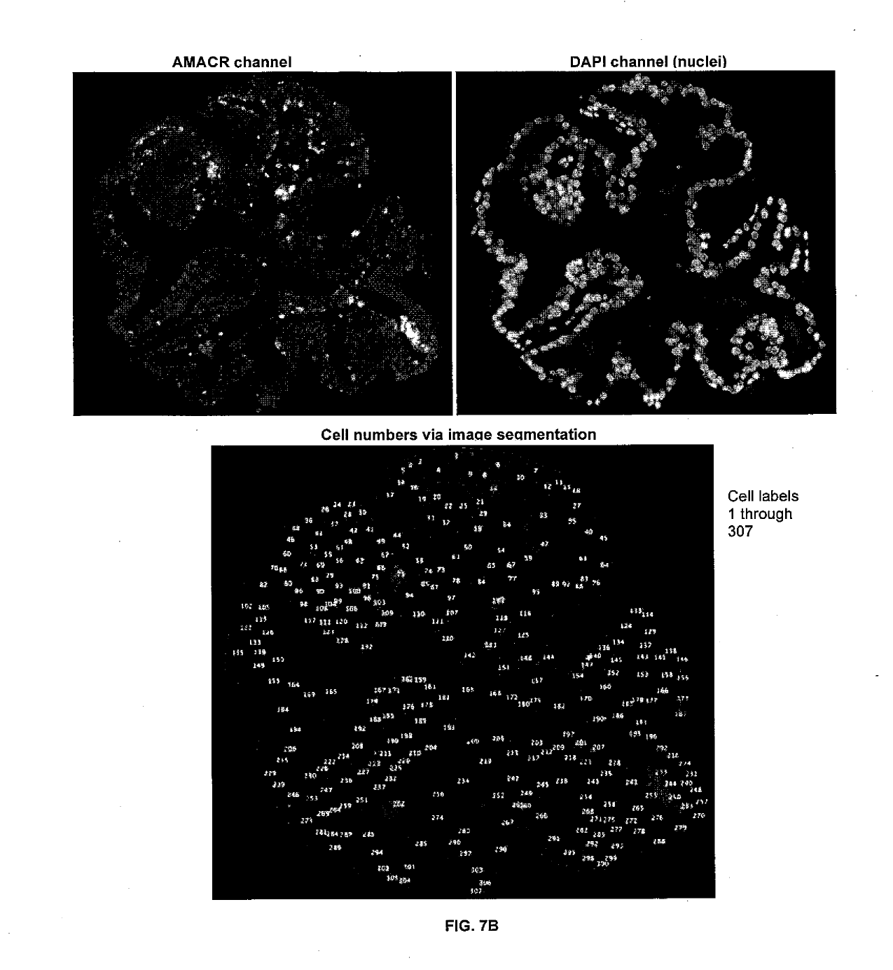

The steps of preparing the sample, scanning the images and performing the operations of FIG. 4 are shown in the flow chart (500) of FIG. 5. The tissue sample is typically obtained during a biopsy procedure or more extensive surgical removal of a larger tumor, formalin fixed, paraffin embedded, tissue sectioned and placed on a slide (501). The slide with the tissue section is then further prepared by deparaffinization antigen retrieval (502), fluorescently labeled for one or more biomarkers and coverslipped (503). Alternatively, hematoxylin (H), which is not typically fluorescent, can be used to label the tissue section prior to immunofluorescent labeling. The tissue is fluorescently scanned at appropriate resolution (typically with a 10×, 20× or 40× high NA dry objective) and the images are knitted together (504) as in the example image 400 in FIG. 4. If already also labeled with H, the tissue is also scanned in bright field for H. If further fluorescent labeling is required (506), the coverslip is removed, the previous fluorescent labels are stripped off (505) and the workflow returns to step 503 for repeated fluorescent labeling until all fluorescent labeling and scanning is completed. If no more fluorescent labeling and scanning is required, and further bright field (clinical) labeling is required (that is if H was not already added or E or other clinical labels are needed), the coverslip is removed (505) and clinical labels are added (507), the coverslip is replaced, the slide is scanned in bright field (509) to produce the clinical image (e.g., H&E image 410 in FIG. 4), and the fluorescent and clinical images are displayed side-by-side, as shown in pairs 400 (F) and 410 (H&E), and 420 (H&E) and 430 (F) in FIG. 4. Automated histocytometry (cell-by-cell image segmentation and measurement analysis of all of the biomarker quantities in each cell and each cell compartment) is then carried out (511), results of which are demonstrated in image 421 and data 450 in FIG. 4, data 605 in FIG. 6, image 704 (right) in FIG. 7A, the bottom image of FIG. 7B, and the data of FIG. 12. Pan, scroll and zoom functions, which work synchronously to produce the same effects on both the fluorescence and bright field images (512), are available to the pathologist to aid in locating the tumor or other interesting tissue region, for manual annotation (421 in FIG. 4; 603 in FIGS. 6; 701, 702, 703, 704 and 705 in FIG. 7A, of which 705 is further demonstrated in FIG. 7B). The automated image cytometry (511) is then automatically displayed and refreshed (e.g., 450 in FIG. 4) for each annotation (514). The data for each annotation is stored by the operator/pathologist on the computer hard drive for compilation into the clinical report.

Further examples of synchronous zooming are demonstrated in the imaging sequence 600 of FIG. 6. An area consisting of about ¼ of the cross-section of a prostate is shown in the image (knitted together from hundreds of fields of view) in the fluorescence and H&E image pair of 601. The area of 602 is then zoomed into the middle pair of images and further zoom is carried out on the region of 603 to create the image pair at the bottom. The image cytometry of annotated (segmented) region 604 is then represented by data plot 605.

Further examples of parts of the workflow (700) are shown in FIG. 7A, where biomarkers are analyzed in the tumor region 705 as well as nearby reactive stroma in regions 701, 702, 703 and 704, with cell-by-cell image segmentation of 704 also shown (right). The tumor region 705 of FIG. 7A is further shown extracted in FIG. 7B, which shows images of the AMACR fluorescence biomarker label (top left), the nuclear channel (DAPI, top right) and the cell numbers created from image segmentation in the histocytometry step (bottom).

Side-by-Side H&E and Immunofluorescence. To immunofluorescently label the prostate tissue with antibodies validated using IHC, we developed a high contrast, low background FFPET-IF protocol to produce whole slide images that exhibit low autofluorescence. Briefly, five micron thick FFPET serial sections are “baked” overnight at 580 C, deparaffinized in xylene and graded series of alcohols before “epitope retrieval” in a household pressure cooker. The tissue sections are double-labeled using rabbit and mouse primary antibodies followed by species-specific Alexafluor™ conjugated secondary antibodies and counterstained with the fluorescent nuclei stain 4′,6-diamidino-2-phenylindole (DAPI). We've seen the protocol work equally well with more than 20 different antibodies using both prostate and breast cancer tissue, with no changes in reagents except the antibody concentrations. The fluorescently-labeled sections are then scanned on our histocytometer, decoverslipped and restained using the standard H&E protocol. FIGS. 7A-B show example images labeled for prostate cancer marker AMACR, stroma marker FHL1 and DAPI, along with a view of the H&E image of the same tissue section and histocytometry by CyteSeer; data in Table 3 is an example of a display of measurements the pathologist sees on the same screen as the H&E and immunofluorescent images, examples of which are also shown.

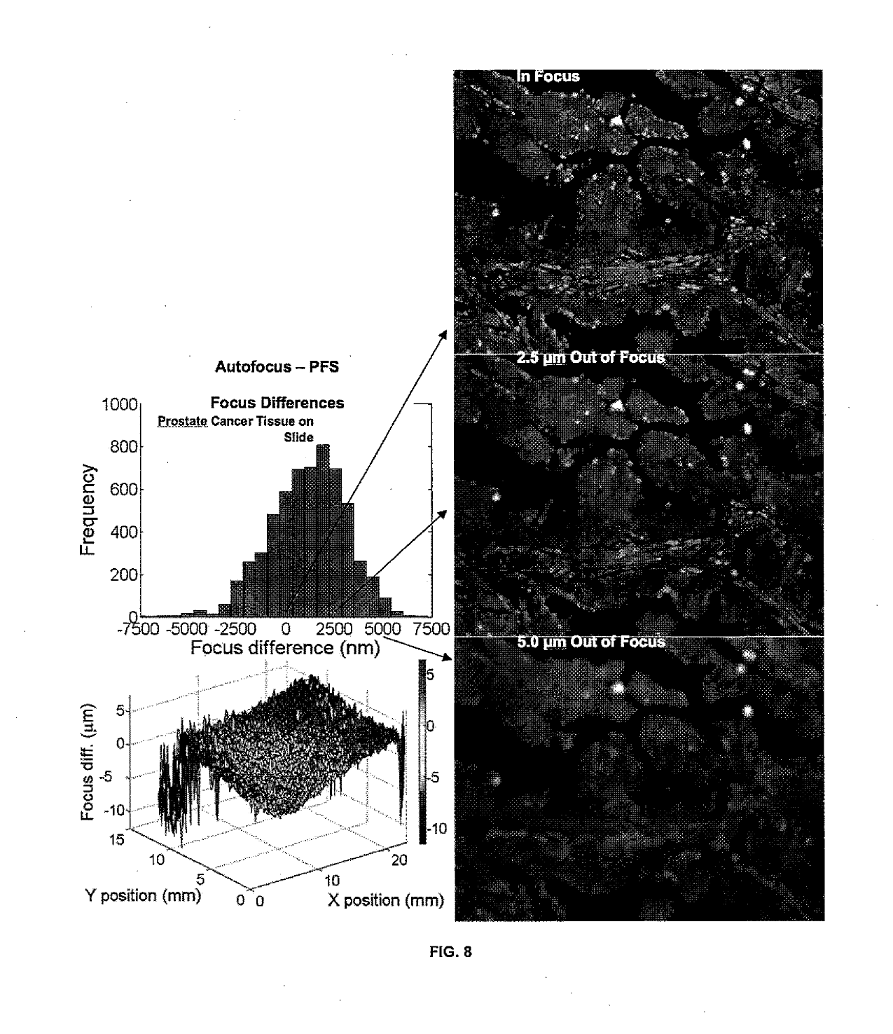

Scanning and Autofocus on Thousands of Fields of View per Slide. To demonstrate use of the tools described herein for scanning a FFPET-IF histological section of human prostate cancer, we scanned the slide on an automated microscopy system built around a Nikon Ti-E microscope, which included the Nikon Perfect Focus System to perform reflective positioning as a method to maintain focus (PFS, see http://www.microscopyu.com/tutorials/flash/focusdrift/perfectiocus/index.html). To the Nikon system, we added our own image-based autofocus to measure the errors of using reflective positioning for autofocus. The error range is about ±7 μm than for cells cultured on the coverslip as shown in FIG. 8. The 3D plot of focus errors exhibits tilt because the coverslip is not parallel to the slide, which contributes to the larger range of errors. Conversely, focus tracking is compromised at the left and right corners of the scan region in the 3D plot of focus differences because those areas of the slide contained no tissue.

Image-based autofocus reliably delivered the best focused images in “normal” fields of view when a difference could be discerned by eye. “Normal” fields of view are defined as those where is tissue present and there is no out-of-focus debris. These data demonstrate that autofocus delivers the best focused images on “normal” fields of view that have tissue present and are not compromised by debris out of the focal plane of the target tissue. Reflective positioning kept track of the focal plane, even without any tissue present, within the 10-μm search range of autofocus of the fields.

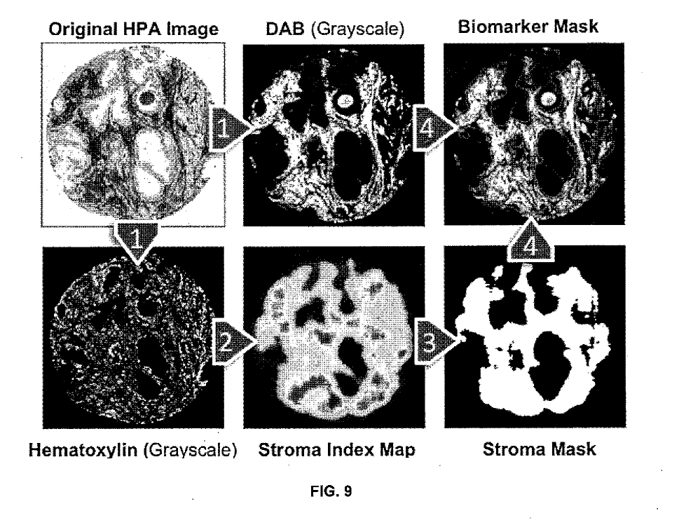

Pattern Analysis in the Adjacent Stroma Regions of Cancer Tissue Sections. Using the Human Protein Atlas (HPA) we identified antibodies for formalin-fixed paraffin-embedded tissue (FFPET), that have been made against >50% of the gene products in the 114 gene Diagnostic Classifier. HPA is an open resources of more than 9.1 million manually annotated immunohistochemistry (IHC) images, generated from >11,000 antibodies targeting proteins from >8,000 protein-coding genes. The HPA includes 67 normal cell types from 144 individuals and 25 different cancer cell types from 216 different tumors with pathologist annotation for each antibody (Berglund, L., et al., “A genecentric Human Protein Atlas for expression profiles based on antibodies,” Mol Cell Proteomics 7, 2019-2027, 2008; Ponten, F., Jirstrom, K. & Uhlen, M., “The Human Protein Atlas—a tool for pathology,” J Pathol 216, 387-393, 2008). For each HPA antibody there are a total of 27 high-resolution tissue microarray (TMA) images (3 normal samples and 12 prostate cancer patients in duplicate) in HPA and there are currently >2,300 high-resolution (9 megapixel) images available from the 87 HPA antibodies that correspond to 62 of Dr. Mercola's Diagnostic Classifier's 114 predicted stroma-specific genes. To systematically identify and quantitatively analyze these stroma-specific images, we use the pattern analysis as demonstrated in FIG. 9 (R Survival. R Development Core Team, v2.15, ISBN 3-900051-07-0, http://www.R-project.org). When mining images from databases such as the HPA, images identified as having stroma-specific biomarkers are independently confirmed after automated analysis by a pathologist. Antibody-based proteomics use protein-specific antibodies to functionally explore the proteome. The tools and methods described herein enable the mining of databases such as the HPA by applying image analysis to validate prostate cancer biomarkers. The value of this approach will increase as the number of prostate tissue images labeled with HPA antibodies increases (rate of increase has been >80,000 per year).

In addition to identifying the images that are stroma-specific, this method quantifies changes in pixel intensity in a distance-dependent manner from tumor foci (see FIG. 9) using color unmixing. The DAB channel (brown) contains the biomarker information (pixel density), and the hematoxylin channel (blue) contains the nuclear features, and FIG. 9 shows example corresponding greyscale images. The nuclei are segmented, and the binary image is used for separating the stroma from the background (i.e., glands, tumor and other tissue components). The hematoxylin grayscale image is used to identify all characteristic stroma nuclear features (e.g., elongated nuclei with non-smooth edges and reduced nuclear density) and for creating a stroma index map (FIG. 9, right arrow #2), which maps the densities of stroma features for the image by contours. Thresholding is applied to the stroma index map (FIG. 9, right arrow #3), converting it into a stroma mask (white area) which contains the topology of stroma density features as pixel data. The stroma in the hematoxylin density grayscale image is identified using the nuclear features only, independent of the biomarker channel. Finally, the DAB density grayscale map is thresholded to create a DAB mask (FIG. 9, right arrow #4) of the biomarker label. The stroma mask is used to integrate the density under the DAB mask (FIG. 9, up arrow #4) to calculate the biomarker density. The final step is to quantify DAB pixel density as a function of distance from tumor foci. Regions excluded from the stroma mask are assumed to be either prostate glands or tumors. The final result is a stroma pixel contour map (black lines=tumor foci; colors=contour levels from the gland/tumor foci) that assigns a distance to each stroma pixel (FIG. 10). The in silico analysis time to accomplish this is ˜20 seconds per HPA image. The average pixel density is then be calculated and plotted versus distance (FIG. 10). We use the slope (linear fit) to quantify and compare protein expression changes as a function of distance from the presence of tumor. The positive slope value in FIG. 10 is an example of protein expression increasing with distance from presence of tumor. In the examples of FIGS. 9 and 10, region image segmentation was used to find the larger scale patterns, and image cytometry (cell-by-cell analysis) is utilized (see FIGS. 7A-B) along with it. These gradients of stromal biomarkers can thereby be used to detect the presence of tumor outside of the tissue section and thereby outside of the biopsy.

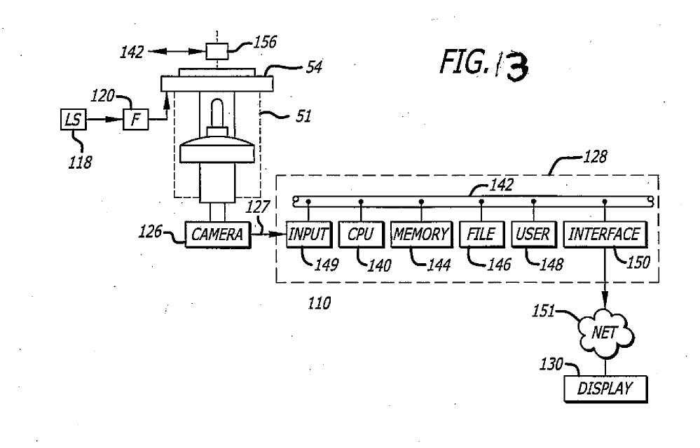

FIG. 13, which is meant for example and not for limitation, illustrates an automated microscopy system adapted to perform automated image cytometry by using tools and executing steps described and illustrated herein. In this regard the system can functions as an automated cytometry system. The system is adapted by addition of programming for the image acquisition and processing, labeling, and presentation steps of this specification to the automated image cytometry programming. The system performs a computer-executed method of automated image cytometry according the Detailed Description and which may be implemented in part in a software program, for example, a program written in the C++ and/or Java programming languages; a counterpart system may be a special purpose computer system designed to execute the method steps of this disclosure. Of course, the method and the programmed computer system may also be embodied in a special purpose processor provided as a set of one or more chips. Further, there may be a program product constituted of a program of computer or software instructions or steps stored on a tangible article of manufacture which is not a network node that causes a computer to execute the method. The tangible article of manufacture may be constituted of one or more real and/or virtual data storage articles such as CDs, DVDs, memory sticks, hard drives, server systems and memory storage devices, and possibly other devices.

In FIG. 13 the automated image cytometry system is constructed and programmed to perform automated image processing, image data management, and image data analysis operations of automated cytometry systems. For example, the instrumentation system may be, or may reside in, or may be associated with a microscopy system 100 including the microscope 51 with the motorized, automatically moveable stage 54 on which a carrier with biological material may be disposed for observation by way of the microscope 51. The carrier may be the multi-well plate 55 discussed above whose wells are disposed in a two dimensional array. For example, and without limitation, the multi-well plate 55 may be a ninety-six well micro-titer plate in each well of which there is biological material that has been cultured, activated, fixed, and stained. A light source 118 provides illumination for operation of the microscope 51 by way of an optical filter 120 and a fiber optic cable 122. The moveable stage 112 may be intermittently or continuously moved to enable the acquisition of one or more magnified images such as are shown in the figures. Images in the field of view of the objective 52 are directed by mirrors and lenses to a high-resolution digital camera 126. The camera 126 obtains and buffers magnified images and transfers them on an interface 127 to a processor 128. The interface 127 may be, for example and without limitation, a universal serial bus (USB). The magnified images are constituted of digital images which may be provided in some standard format comprising an N×M array of pixels to the processor 128. The processor 128 receives the digital images and stores them in image files. The digital images are processed by the processor 128 and output digital images may be provided by the processor 128 for display on an output device with a display 130. The processor 128 also includes programming and processes to process and analyze image content and to create and display charts, graphs, tables, and possibly other means for displaying analysis results.

With further reference to FIG. 13, the processor 128 may be a programmed general purpose digital processor having a standard architecture, such as a computer work station. The processor 128 includes a processing unit (CPU) 140 that communicates with a number of peripheral devices by way of a bus subsystem 142. The peripheral devices include a memory subsystem (MEMORY) 144, a file storage subsystem (FILE) 146, user interface devices (USER) 148, an input device (INPUT) 149, and an interface device (INTERFACE) 150. It is not necessary that the processor 128 be connected directly to the microscope 51; it may receive images produced by the camera 126 from a portable storage device, or by way of a local or wide area network. For example, images may be transported to the processor 128 over the internet.

The bus subsystem 142 includes media, devices, ports, protocols, and procedures that enable the processing unit 140 and the peripheral devices 144, 146, 148, 149, and 150 to communicate and transfer data. Instrumentation 156 such as is illustrated in FIG. 6 may also be coupled to the bus subsystem 142 by a conventional instrumentation interface. The bus subsystem 142 provides generally for the processing unit and peripherals to be collocated or dispersed

The memory subsystem 144 includes read-only memory (ROM) for storage of one or more programs of instructions that implement a number of functions and processes. One of the programs is an automated image process for processing a magnified image of biological material to identify one or more components of an image. The memory subsystem 144 also includes random access memory (RAM) for storing instructions and results during process execution. The RAM is used by the automated image process for storage of images generated as the process executes. The file storage subsystem 146 provides non-volatile storage for program, data, and image files and may include any one or more of a hard drive, floppy drive, CD-ROM, and equivalent devices

The user interface devices 148 include interface programs and input and output devices supporting a graphical user interface (GUI) for entry of data and commands, initiation and termination of processes and routines and for output of prompts, requests, screens, menus, data, images, and results.

The input device 149 enables the processor 128 to receive digital images directly from the camera 126, or from another source such as a portable storage device, or by way of a local or wide area network. The interface device 150 enables the processor 128 to connect to and communicate with other local or remote processors, computers, servers, clients, nodes and networks. For example, the interface device 150 may provide access to an output device 130 by way of a local or global network 151. As a further example, the interface device 150 may provide the automated microscopy system with access to remote databases by way of the local or global network.

In the context of the automated image cytometry system of FIG. 13, those skilled in the art will realize that FIGS. 4 and 5 can be implemented in a in a software program containing instructions for operating the automated image cytometry system shown in FIG. 13 to perform a computer-executed method according to this disclosure.

The scope of patent protection afforded the novel tools and methods described and illustrated herein may suitably comprise, consist of, or consist essentially of any combination of the elements described above. Further, the novel tools and methods disclosed and illustrated herein may suitably be practiced in the absence of any element or step which is not specifically disclosed in the specification, illustrated in the drawings, and/or exemplified in the embodiments of this application. Moreover, although an invention has been described with reference to presently preferred embodiments, it should be understood that various modifications can be made without departing from the spirit of the invention.

TABLE 1
Common Stains of Tissues used for Classical Subjective Diagnoses

| | | | | Red blood | Collagen | |
|---|---|---|---|---|---|---|
| Stain | Common use | Nucleus | Cytoplasm | cell (RBC) | fibers | Specifically stains |
| Haematoxylin | General staining | Blue | N/A | N/A | N/A | Nucleic acids-blue |
| | when paired | | | | | ER (endoplasmic reticulum)-blue |
| | with eosin | | | | | |
| | (i.e. H&E) | | | | | |
| Eosin | General staining | N/A | Pink | Orange/red | Pink | Elastic fibers-pink |
| | when paired with | | | | | Collagen fibers-pink Reticular fibers-pink |
| | haematoxylin | | | | | |
| | (i.e. H&E) | | | | | |
| Toluidine blue | General staining | Blue | Blue | Blue | Blue | Mast cells granules-purple |
| Masson's | Connective tissue | Black | Red/pink | Red | Blue/green | Cartilage-blue/green |
| trichrome | | | | | | Muscle fibers-red |
| stain | | | | | | |
| Matlory's | Connective tissue | Red | Pale red | Orange | Deep blue | Keratin-orange |
| trichrome | | | | | | Cartilage-blue Bone matrix-deep blue |
| stain | | | | | | Muscle fibers-red |
| Weigert's | Elastic fibers | Blue/black | N/A | N/A | N/A | Elastic fibers-blue/black |
| elastic stain | | | | | | |
| Heidenhain's AZAN | Distinguishing cells | Red/purple | Pink | Red | Blue | Muscle fibers-red |
| trichrome stain | from extracellular | | | | | Cartilage-blue Bone matrix-blue |
| | components | | | | | |
| Silver stain | Reticular fibers, | N/A | N/A | N/A | N/A | Reticular fibers-brown/black |
| | nerve fibers, fungi | | | | | Nerve fibers-brown/black |
| Wright's stain | Blood cells | Bluish/purple | Bluish/gray | Red/pink | N/A | Neutrophil granules-purple/pink |
| | | | | | | Eosinophil granules-bright red/orange Basophil |
| | | | | | | granules-deep purple/violet Platelet |
| | | | | | | granules-red/purple |
| Orcein stain | Elastic fibres | Deep blue | N/A | Bright red | Pink | Elastic fibres-dark brown |
| | | | | | | Mast cells granules-purple Smooth |
| | | | | | | muscle-light blue |
| Periodic | Basement | Blue | N/A | N/A | Pink | Glycogen and other carbohydrates-magenta |
| acid-Schiff | membrane, | | | | | |
| stain (PAS) | localizing | | | | | |
| | carbohydrates | | | | | |

TABLE 2
FDA-approved scoring system for HER2/neu:

| Score | Definition |
|---|---|
| 0 | No immunostaining |
| 1+ | Weak immunostaining <30% of cells |
| 2+ | Complete membranous staining >10% of cells |
| 3+ | Uniform intense membranous staining >30% of cells |

TABLE 3

| Region | Mean AMACAR | Mean HL1 | Cell Count |
|---|---|---|---|
| Tumor | 11.93 | 1.40 | 341 |
| Adjacent | 2.59 | 6.32 | 27 |
| Near | 4.20 | 15.32 | 23 |
| Far | 5.36 | 14.59 | 35 |
| Very Far | 4.59 | 12.32 | 53 |

1. A method of operating an automated microscopy system for measuring biomarker labels, comprising:
labeling tissue sections with fluorescent labels for molecules;
an automated microscopy-executed step of acquiring images of the fluorescently-labeled tissue sections;
an automated microscopy-executed step of labeling the same tissue sections with classical diagnostic bright field dyes;
an automated microscopy-executed step of scanning the classically-labeled tissue sections to acquire images of them; and
an automated microscopy-executed step of generating and displaying measurements of biomarker labels.

2. The method of claim 1, further including an automated microscopy-executed step of displaying the fluorescent images and the bright field images together.

3. The method of claim 1, an automated microscopy-executed step of generating cell-by-cell measurements.

4. The method of claim 3, wherein the measurements include distances of cells from a region or regions within the tissue, further including an automated microscopy-executed step of generating gradients from the distances for detecting the presence of tumor outside the tissue section.

5. The method of claim 2, further including an automated microscopy-executed step of displaying the images and the measurements of the biomarkers together.

6. The method of claim 2, further including an automated microscopy-executed step of displaying multiple labels and stains together in response to a user selection.

7. The method of claim 6, further including an automated microscopy-executed step of associating the multiple labels in subpopulations of cells with specific combinations of biomarkers.

8. The method of claim 2, further including an automated microscopy-executed step of receiving specification of one or more regions of the images for obtaining biomarker measurements on those regions.

9. The method of claim 8, further including an automated microscopy-executed step of comparing the measures of biomarkers derived from different regions of the tissue.

10. The method of claim 2, further including an automated microscopy-executed step of displaying the images and measures from the on-slide standard for normalizing the measures of the biomarkers of other tissues.

11. The method of claim 10, further including an automated microscopy-executed step of comparing the measures of biomarkers in the on-slide standard with measures of biomarkers from the on-slide standards from other slides in order for normalizing the measurements across slides.

12. The method of claim 11, further including an automated microscopy-executed steps of comparing the measures of biomarkers from on-slide standards on slide to the measures of the on-slide standards on a reference slide or reference slides and normalizing measures of biomarkers.

13. The method of claim 12, further including an automated microscopy-executed step of accessing the measures of the on-slide standards on the reference slides through a web site.

14. The method of claim 12, wherein a database of patient laboratory results of measurements of biomarkers utilizes the measures of the on-slide standards as a reference to compare and normalize measures of biomarkers on tissues stored in the database.
